(19) United States
(12) Patent Application Publication
(10) Pub. No.: US 2007/0134312 A1
(43) Pub. Date: Jun. 14, 2007

(54) Liposomes for treatment of multiple myeloma

(76) Inventor: Mohamad A. Hussein, Brecksville, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/519,343

(22) Filed: Sep. 12, 2006

Related U.S. Application Data
(60) Provisional application No. 60/716,447, filed on Sep. 12, 2005.

(51) Int. Cl. A61K 39/395; A61K 9/127; A61K 31/704

(52) U.S. Cl. 424/450; 514/34; 424/144.1

(57) ABSTRACT

A method for treating multiple myeloma in newly diagnosed or previously treated patients is described. The method comprises administering a composition consisting of a combination of chemotherapeutic agents of an anthracycline antibiothic, preferably entrapped in a liposome, dexamethasone, and thalidomide, and, optionally, a reduced dose of vincristine.

### CROSS-REFERENCE TO RELATED APPLICATIONS

This application claims the benefit of U.S. Provisional Application No. 60/716,447, filed Sep. 12, 2006, incorporated herein by reference in its entirety.

### TECHNICAL FIELD

The subject matter described herein relates to a method of treating multiple myeloma by administering a combination of chemotherapeutic agents of an anthracycline entrapped in a liposome, dexamethasone, and thalidomide, and, optionally, a reduced dose of vincristine.

### BACKGROUND

Multiple myeloma represents a malignant proliferation of plasma cells. The disease results from the uncontrolled proliferation of plasma cells derived from a single clone. The tumor, its products, and the host response to it result in a number of organ dysfunctions and symptoms of bone pain or fracture, renal failure, susceptibility to infection, anemia, and other symptoms.

An estimated 15,270 new cases of multiple myeloma will be diagnosed in 2005, and an estimated 11,070 patients will die of their disease (Jemal, A. et al., CA Cancer J. Clin., 54(1):8-29 (2004)).

Treatment of multiple myeloma with melphalan plus prednisone, which produces response rates of 50% to 60%, has been a traditional first-line therapy. The combination of vincristine and doxorubicin (Adriamycin™), both administered as a continuous 96-hour infusion via a central line, plus intermittent high-dose dexamethasone (VAD regimen) is another common treatment regimen for patients with newly diagnosed or refractory multiple myeloma (Hussein, M. A., Oncologist; 8 Suppl 3:3945 (2003)). Response rates of 55% to 84% have been reported in newly diagnosed patients treated with the VAD regimen, with median remission duration of approximately 18 months (Hussein, M. A., supra).

More recently, liposomal doxorubicin has been used in the VAD regimen, where doxorubicin entrapped in long-circulating liposomes (Doxil®) replaces the free form of the drug in the VAD combination (Hussein, M. A. et al., Seminars in Oncology, 31 (Suppl 13):147-160 (2004); Hussein, M. A. Let al. Cancer, 95(10):2160-2168 (2002)). Liposomal doxorubicin has a prolonged blood half-life relative to the free form of the drug, thus allowing increased exposure of the myeloma cells to the drug.

The conventional VAD regimen has also been modified by addition of thalidomide, to treat the patients with a combination of doxorubicin, in free form or in liposome-entrapped form, vincristine, and dexamethasone (DVD-T or T-VAD) (Hussein M., Oncologist; 8 Suppl 3:39-45 (2003); Zervas, K. et al., Annals of Oncology, 15:134-138 (2004); Ahmad, I. et al., Bone Marrow Transplantation, 29:577-580 (2002)).

Despite these various treatment approaches, there remains a need in the art for a treatment regimen that improves the quality of response in newly diagnosed multiple myeloma patients and the response rate and quality of response in relapsed/refractory multiple myeloma patients.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method for treating multiple myeloma is provided. The method comprises administering a combination of chemotherapeutic agents consisting essentially of an anthracycline in liposome-entrapped form, dexamethasone, thalidomide, and a dose of vincrstine less than a recommended dose for treatment of multiple myeloma.

In one embodiment of the method, dexamethasone is administered orally at decreased frequency of administration, relative to the frequency recommended on the product label for treatment of multiple myeloma or relative to the frequency recommended in the literature for treatment of multiple myeloma.

In another embodiment, the agents are administered prior to autologous stem cell transplant.

In yet another embodiment, the agents are administered prior to or concurrent with an induction therapy regimen to mobilize stem cell production.

In one embodiment, the liposome-entrapped anthracycline is liposome-entrapped daunorubicin. In another embodiment, the liposome-entrapped anthracycline is liposome-entrapped doxorubicin.

In still another embodiment, the liposome-entrapped doxorubicin is comprised of liposomes having an external coating of a hydrophilic polymer. An exemplary hydrophilic polymer, in one embodiment, is poly(ethylene glycol).

The liposomes, in another embodiment, comprise a ligand for targeting the liposomes to a B-cell or a T-cell. Exemplary ligands include, but are not limited to, an anti-CD19 antibody, an anti-CD20 antibody, an anti-CD22 antibody, an anti-CD4 antibody, and an anti-CD8 antibody.

In another embodiment, the thalidomide is administered at a dose of at least about 50 mg/day.

In another embodiment, dexamethasone is administered orally. In still another embodiment, dexamethasone is administered at a dose of at least about 40 mg.

In another embodiment, the combination of agents is administered once every four weeks for at least about three months. In an alternative embodiment, the combination of agents is administered once every four weeks for at least about six months. In yet another embodiment, upon completion of the treatment regiment, for example upon completion of a three or six month treatment period wherein the combination was administered once every four weeks, the method further comprises administering prednisone.

In another aspect, an improvement in a method of treating multiple myeloma by treatment with liposome-entrapped doxorubicin, vincristine, dexamethasone, and thalidomide is provided, where the improvement comprises administering, in the absence of vincristine, a combination of chemotherapeutic agents consisting essentially of doxorubicin in liposome-entrapped form, dexamethasone, and thalidomide.

In still another aspect, a method for treating multiple myeloma is provided, where administering, in the absence of vincristine, doxorubicin in liposome-entrapped form, dexamethasone, and thalidomide.

In yet another aspect, a method for treating multiple myeloma comprised of administering a combination of chemotherapeutic agents consisting essentially of doxorubicin entrapped in liposomes, the doxorubicin administered intravenously at a dose of at least about 40 mg/m²; dexamethasone administered orally at dose of at least about 40 mg, thalidomide administered orally at a dose of at least about 50 mg is provided.

In another aspect, a method for treating multiple myeloma is provided, the method comprising administering over a 28-day treatment cycle a combination of chemotherapeutic agents consisting essentially of (a) doxorubicin entrapped in liposomes, administered intravenously at a dose of at least about 40 mg/m² on day one of the treatment cycle; (b) dexamethasone, administered orally at dose of at least about 40 mg on days 1-4, 9-12 and 17-20 of the treatment cycle; and (c) thalidomide administered orally at a dose of at least about 50 mg per day; and repeating the administering between 4-12 times.

In addition to the exemplary aspects and embodiments described above, further aspects and embodiments will become apparent by reference to the drawings and by study of the following descriptions.

### BRIEF DESCRIPTION OF THE DRAWINGS

FIG. 1 is a bar graph showing the quality of response following treatment of multiple myeloma patients with Doxil, vincristine, dexamethasone, with or without thalidomide (DVd-T and DVd, respectively). The fraction of patients with a best response, corresponding to the patients with a complete response or a non-complete response, and a good response, corresponding to the patients with a partial response or stable disease.

FIGS. 2A-2B are graphs showing the survival probability as a function of progression free survival (FIG. 2A) or overall survival (FIG. 2B) in multiple myeloma patients treated with DVd-T and with DVd.

FIGS. 3A-3B are graphs showing the survival probability as a function of progression free survival (FIG. 3A) or overall survival (FIG. 3B) in multiple myeloma patients treated with DVd-T or with DVd and exhibiting a best response (patients with a complete response or a non-complete response) or a good response (the patients with a partial response or stable disease).

FIG. 4 is a graph showing the survival probability as a function of progression free survival in multiple myeloma patients treated with DVd-T, with vincristine at a dose of 2 mg (“no vincristine reduction”) or with a 50% reduction in vincristine dose.

### DETAILED DESCRIPTION

I. Method of Treatment

The method for treating multiple myeloma is based on the finding that removing vincristine from, or reducing the dose of vincristine in, the conventional “DVd-T” treatment regimen consisting of doxorubicin (free form or liposome entrapped), vincristine, dexamethasone, and thalidomide, provides an improved therapeutic response with a reduction in adverse events. More generally, the method relates to a treatment regimen consisting essentially of an anthracycline antibiotic, in free form or in liposome-entrapped form, dexamethasone, and thalidomide, in the absence of vincristine or in the presence of a reduced dose of vincristine.

As used herein, a “reduced dose” of vincristine refers to a dose that is at least about 25% lower, more preferably at least about 35% lower, and still more preferably at least about 50% lower than the recommended dose for treatment of multiple myeloma. Vincristine has been included in many multiple myeloma based regimens, and in the DVd-T regimen is given at about 2 mg as an infusion over 1-3 hours.

In treatment regimens for multiple myeloma and other cancers, time-to-event endpoints are commonly used as major endpoints in clinical trials. Such endpoints include overall survival (OS), time to progression (TTP) (also referred to as progression-free survival, PFS), disease-free survival (DFS) (also referred to as relapse-free survival, RFS), time to treatment failure (TTF) and so on. PFS is generally refers to the length of time during and after treatment that the cancer does not grow. Progression-free survival includes the amount of time patients have experienced a complete response or a partial response, as well as the amount of time patients have experienced stable disease. A remission, complete remission or complete response refers to an absence of cancer cells after treatment, for at least about six months. Partial remission, or partial response, indicates there has been a decrease in tumor size, or in the extent of cancer in the body, after treatment. The definition of “partial” is different for every cancer. Overall survival refers to the total amount of time that a patient survives following treatment, the total time usually reported from the time since diagnosis or treatment. Example 1, discussed below, sets forth the specifics for these end points used in the supporting studies herein.

In a study supporting the treatment method, patients with newly diagnosed or previously treated, relapsed refractory multiple myeloma were treated with a combination of doxorubicin entrapped in pegylated liposomes (Doxil®, “D”), vincristine (“V”), dexamethasone (“d”), and thalidomide (“T”) (“DVd-T”), as set forth in Example 1. Of 102 eligible patients, the treatment regimen provided quality response (complete response and very good partial response) of 49% in the newly diagnosed patient group and 45% in the previously treated patient group.

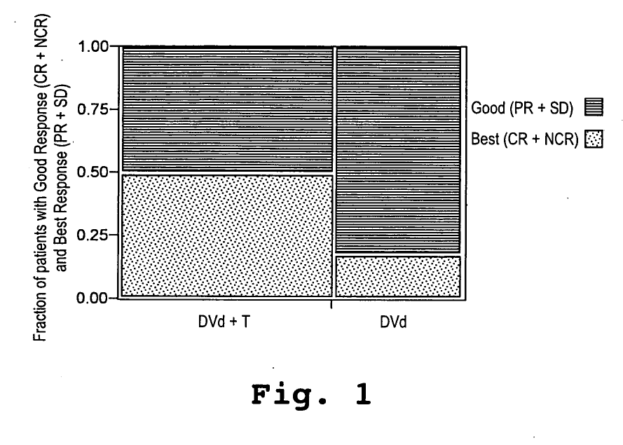

In some patients in the study, thalidomide was omitted from the treatment regimen. FIGS. 1, 2A, 2B, 3A, and 3B compare the response of patients treated with DVd or with DVd-T, as further detailed in Example 1.

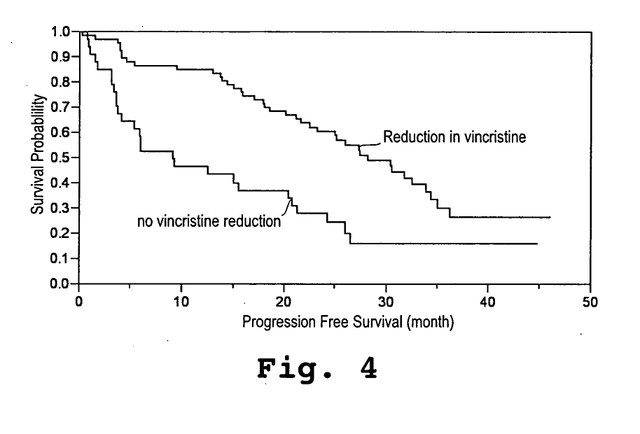

Patients exhibiting a grade 3 or 4 adverse event in the study received a modified treatment regimen, where the dose of vincristine was reduced by about 50% of the initial starting dose, or where vincristine was eliminated from the treatment regimen. FIG. 4 compares the survival probability as a function of progression free survival, in months, for patients treated with the full 2 mg dose of vincristine to the patients receiving a reduced dose of vincristine (1 mg). The reduction of the vincristine dose continued to benefit patients with a continued significant improvement in the progression free survival, as compared to the group that received the full dose. A similar result is expected for patients treated with a regimen of an anthracycline antibiotic, dexamethasone, and thalidomide.

This surprising results illustrates that a beneficial effect in the treatment of multiple myeloma patients was found by reducing or eliminating vincristine from the DVd-T regimen. It was a surprise to discover this positive impact from reducing or eliminating vincristine was not related to the dose of the thalidomide, i.e., patients with lower dose or no vincristine in their regimen did not receive a higher dose or longer therapy with thalidomide.

While the study in example 1 uses the anthracycline antibiotic doxorubicin, it will be appreciated that the treatment regimen contemplates other anthracycline drugs, such as daunorubicin, epirubicin, idarubicin, and mitoxantrone. It will also be appreciated that the drug can be administered in free form or in liposome-entrapped form.

Preparation of liposomes containing an entrapped drug are well described in the literature. The liposomes may additionally include a surface coating of a hydrophilic polymer, exemplified, but not limited to poly(ethylene glycol). Other hydrophilic polymers include polyvinylpyrrolidone, polymethyloxazoline, polylactic acid, polyglycolic acid, and others described in U.S. Pat. No. 5,631,018. The hydrophilic polymer recited in this patent are incorporated by reference herein.

In one embodiment, the polymer-coated liposomes additionally include a targeting ligand, such as an antibody or antibody fragment, to target the drug-loaded particles to a specific site. Preparation of liposomes having targeting ligands attached to the distal end of liposome-attached polyethylene glycol chains is described, for example in U.S. Pat. Nos. 6,316,024; 6,326,353; 6,056,973. The targeting ligand can be one having binding for a cell receptor implicated in multiple myeloma or plasma cell neoplasm, such as cells of B-cell or T-cell lineage. Exemplary antibodies include, but are not limited to, anti-CD19, anti-CD20 or anti-CD22, for specific binding to a B-cell antigen; and anti-CD4 or anti-CD8 for binding to a T-cell antigen.

The treatment regimen comprises administering to a patient diagnosed with multiple myeloma a combination consisting essentially of an anthracycline antibiotic, dexamethasone, and thalidomide, in the absence of vincristine or with a reduced dose of vincristine. It will be appreciated that the combination can include supportive care measures, such as the addition of prophylactic suppressive antibiotics, antivirals, growth factors for low baseline white blood count as well as low dose aspirin, or other measures determined necessary by a supporting physician to ameliorate symptoms associated with the treatment regimen.

In another embodiment, the treatment method is provided to a patient prior to autologous stem cell transplant, or prior to or concurrent with an induction therapy regimen to mobilize stem cell production. Multiple myeloma patients are treated with the treatment regimen consisting of an anthracycline antibiotic, dexamethasone, thalidomide, in the absence of vincristine or with a reduced dose of vincristine. Patients that achieve a complete response, a very good partial response, or a partial response then undergo autologous peripheral blood stem cell transplantation, or an induction therapy to mobilize stem cell production.

### II. EXAMPLES

The following examples are illustrative in nature and are in no way intended to be limiting.

### Example 1

### Treatment of Multiple Myeloma Patients

A study was initiated in newly diagnosed and relapsed/refractory multiple myeloma patients. One hundred two patients were enrolled for treatment with pegylated liposomal doxorubicin (Doxil®), vincristine, dexamethasone, and thalidomide (Dvd-T), according to the treatment regimen described below. Fifty-three (53) patients were newly diagnosed with multiple myeloma (Group A) and forty-nine (49) had relapsed/refractory disease (Group B).

Descriptive statistics for demographic and baseline variables appear in Table 1. The overall median age was 62.9 years with Group A patients having a 15 significantly lower median age than Group B patients. With respect to gender and race the two groups were similar. Even though not significant, the Group B patients tended to have a more advanced stage of disease (by SWOG criteria) than the Group A patients. The median β₂-microglobulin was higher in the Group B patients, whereas median absolute neutrophil count (ANC) and platelets were higher in the Group A patients. Cytogenetic analysis was available for 88 patients in both groups with 16 patients showing abnormal results. The abnormality rate was the similar in both groups (Table 1).

TABLE 1
Descriptive statistics for Group A and Group B Patients

| | Group A | | Group B | | |
|---|---|---|---|---|---|
| Variable | (n = 53) | | (n = 49) | | p-value ** |
| Age * | 60.5 | (51.3,67.1) | 65.3 | (57.2, 71.1) | 0.046 |
| Gender (% F) | 47 | | 39 | | 0.39 |
| Race (% W) | 75 | | 86 | | 0.19 |
| Time from | 1.4 | (0.6, 6.6) | 28.0 | (15.7, 51.9) | <0.0001 |
| diagnosis to | | | | | |
| start of | | | | | |
| study (mo) | | | | | |
| Stage (1, | 79, 21 | | 62, 38 | | 0.19 |
| 2 vs 3, 4)% | | | | | |
| β₂- | 3.6 | (2.2, 4.7) | 4.6 | (2.7, 8.5) | 0.008 |
| microglobulin | | | | | |
| Absolute | 3.1 | (2.1, 4.9) | 2.5 | (1.7, 3.2) | 0.019 |
| neutrophil | | | | | |
| count | | | | | |
| Platelets | 213 | (181, 274) | 139 | (81, 224) | <0.0001 |

| | | | |
|---|---|---|---|
| Abnormal | 20 | 17 | 0.72 |
| Cytogenetic | | | |
| Result (%) | | | |
| Response (%) | 36, 13, 38, 8, 6 | 21, 26, 40, 13, 0 | 0.078 |
| (CR, NCR, | | | |
| PR, SD, PD) | | | |

* Median (IQR) for continuous variables.
** Wilcoxon test for continuous variables, Chi-square for categorical variables. SD = stable disease; PD = patient death.

Treatment Regimen

The patients in Group A and Group B were treated as follows. On day 1 Doxil® (“D”) was given at 40 mg/m² as a short intravenous infusion over 1 to 3 hours; vincristine (“V”) at 2 mg as a short intravenous infusion over 1 to 3 hours; and dexamethasone (d) at 40 mg daily orally for 4 days. Thalidomide was given at 50 mg per day orally. The thalidomide dose was increased if tolerated by 50 mg/d every week, not to exceed 400 mg a day. The DVd regimen was repeated every four weeks, for a minimum of six cycles and two cycles beyond best response. Following the achievement of best response patients were maintained on prednisone 50 mg every other day and the maximal tolerated dose of thalidomide until disease progression or intolerance.

A separate group of patients was treated with the DVd regimen absent thalidomide.

Supportive Care

The use of bisphosphanates and myeloid and erythropoietic factors was allowed as per the accepted standard of care. The protocol permitted the use of low-dose aspirin at 81 mg daily, amoxicillin at 250 mg twice daily, and acyclovir at 400 mg twice daily. Granulocyte and granulocyte monocyte growth factors were given on day 1 of therapy if the total white blood cell count was not greater than or equal to 5×10⁹/L. I-glutamine was given to patients who developed upper or lower extremity cramping of any severity.

Baseline and Outcome Assessment

Patients were evaluated within 28 days before study entry. Monitored myeloma parameters included β₂-microglobulin, serum albumin, lactate dehydrogenase, serum protein electrophoresis, 24 hour urine collection for total protein and urine protein electrophoresis, and myeloma typing of serum and urine. Laboratory parameters were assessed before each cycle of therapy and at four weeks after the initiation of the maintenance regimen. The Southwest Oncology Group (SWOG) staging system was used for myeloma staging. Serum vitamin B12, red blood cell folate, methylmalonic acid, and serum homocysteine levels were measured at baseline. Bone marrow aspiration, biopsy, cytogenetic analysis, and complete bone survey were performed for all patients at baseline. A baseline echocardiogram or multiple-gated acquisition scan was performed for all patients.

Assessment of response and toxicity included monthly history, physical examination, and laboratory tests in the form of complete blood cell count, complete metabolic profile, serum protein electrophoresis β₂-microglobulin, and 24-hour urine for protein quantitation with urine protein electrophoresis if monoclonal protein was detected in the urine. Monoclonal protein analysis in the serum and urine was performed when the serum and urine protein electrophoresis normalized. Bone marrow aspiration, biopsy, and cytogenetic analysis were performed at the completion of 6 cycles and the completion of chemotherapy if the monoclonal component was not detectable by immune fixation to document complete remission. Patients who completed the chemotherapy portion of the study as outlined herein were given maintenance therapy and evaluated in 1 month and then every 3 months thereafter. Dates of first and best responses were the dates when the parameters for the monoclonal proteins were met and the bone marrow results were available, respectively. The bone survey was performed every 6 months or sooner if clinically indicated. Echocardiography or multiple-gated acquisition scan was performed every 2 cycles after a total dose of 300 mg/m² of pegylated liposomal doxorubicin was reached. All patients were followed up for survival. Toxicity was assessed using the National Cancer Institute Common Toxicity Criteria version 2.0.

Evaluation of Response

Patients were removed from the study in the event of disease progression, unacceptable toxic effects, patient wishes, or the discretion of the investigator.

A complete response (CR) was defined by the total disappearance of the paraprotein in the serum and urine by immunofixation, in addition to less than 5% plasma cells on bone marrow evaluation.

A very good partial response (VGPR), also referred to as a non-complete response (NCR), was defined as a 90% or greater decrease in the serum paraprotein level.

A partial response (PR) was defined as a 50% or greater decrease in the serum paraprotein level and urine levels greater than or equal to a90% reduction of the monoclonal component or a decrease to less than 200 mg/24 h.

A minimal response was defined as a less than 50% decrease in serum or urine paraprotein levels.

Disease progression was defined by the development of two worsening parameters.

Progression free survival (PFS) and overall survival (OS) were calculated from the date of study entry to the date of progression or death. Both the PFS and OS were computed after the date of best response and analyzed with the Cox proportional hazards model to adjust for relevant baseline variables.

All response criteria were verified every four weeks.

Results

After a median follow-up of 28 months, the overall response rates were 97% and 90% for patients with newly diagnosed (Group A) and previously treated (Group B) multiple myeloma, respectively. The rates of quality responses, taken as the sum of the patients who exhibited a complete response (CR) or a very good partial response (VGPR), were 49% for Group A and 45% for Group B. Nineteen (36%) of 53 patients with newly diagnosed disease (Group A) achieved a CR, whereas 10 (20%) of the 49 previously treated patients received a CR. Median time to first and best response was similar in both groups, with a combined median of 1.2 and 4 months, respectively.

The median progression free survival (PFS) for the newly-diagnosed group was 28.2 and 15.5 months for the previously-treated group (p=0.01). Median overall survival was not reached at 50 months of follow-up for the newly diagnosed group of patients, whereas it was 39.9 months for the previously treated group of patients.

Partial response or better (CR, NCR, PR) was noted in 86% and 87% of the patients for the Group A newly-diagnosed and the Group B previously treated patients, respectively. The best response rates, taken as the sum of patients with a complete response or a non-complete response (CR+NCR), for both groups were comparable. However, the CR rate for Group A was higher at 36% vs. 21% for Group B patients.

FIG. 1A is a graph showing the quality of response of patients treated with the DVd+T regimen, compared to patients treated with DVd (absence of thalidomide). When compared to the DVd regimen in the newly diagnosed and relapsed/refractory patients, the addition of the thalidomide to the regimen significantly improved the quality of response, where 50% of the patients receiving DVd-T achieved a best response (CR+NCR), whereas only about 17% of the patients treated with DVd alone achieved a best response (CR+NCR) (p<0.0001).

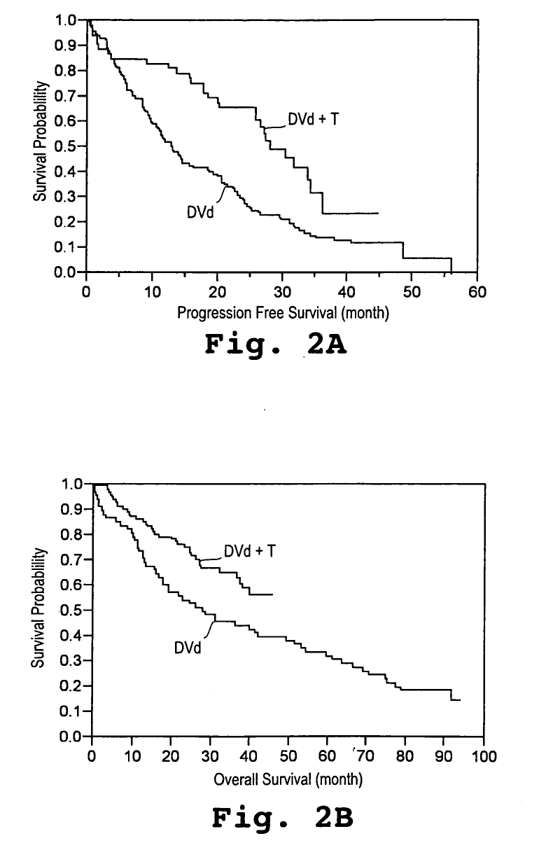

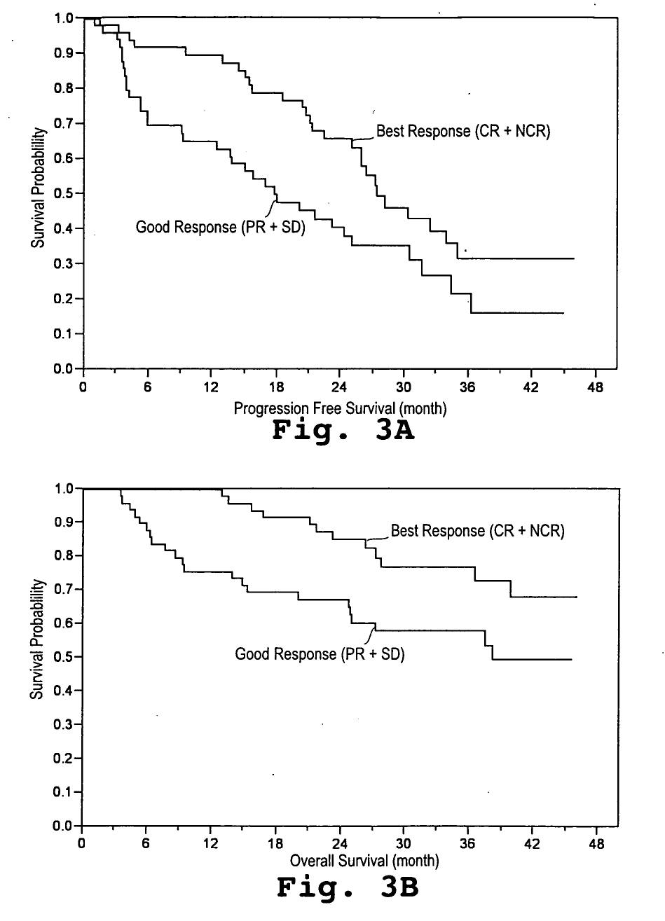

The patient groups were matched for supportive care, demographics, disease stage and bone marrow characteristics, except that the DVd-T group had a higher percentage of bone marrow involvement with plasma cells. As seen in FIGS. 2A-2B, the median progression free survival and median overall survival were significantly improved by the addition of thalidomide to the regimen (28 vs. 13 months; p=0.0003) and (median not reached vs. 27.9 months; p=0.01)). The effect of the quality of response had an impact on median progression free survival and median overall survival, as seen in FIG. 3A-3B.

As seen in FIG. 3A, patients achieving a complete response or a non-complete response (CR or NCR) experienced a progression free survival (PFS) of 27.4 months, as compared to 17.9 months for those achieving partial response or stable disease (PR or SD; p=0.005). FIG. 3B shows that for same two patient sets of CR+NCR and PR+SD, a median was not reached for the CR+NCR patient set yet for the PR+SD patient set, the overall survival was 38.3 months (p=0.007).

Treatment Regimen Modification with Vincristine Dose Reduction

At the start of therapy three patients were neutropenic; however as their multiple myeloma responded their counts normalized. Table 2 gives the adverse events by grade for all of the study patients (105 enrolled, with 102 undergoing treatment). In brief, thrombocytopenia was noted in 19 patients (18%), of whom five had a platelet count of less than 50×10⁹/L. These patients tolerated therapy well, and none required platelet transfusion support. The most common grade 1 and 2 toxic effects that occurred in 20% or more of the patients were palmer planter erythrodysthesia (41%), peripheral neuropathy (84%), constipation (78%), fatigue (60%), and extremity cramps and tremors (20%).

TABLE 2

| Adverse Event | Grade 3/4 | Grade 1/2 |
|---|---|---|
| pneumonia | 12 | NA |
| neutropenia | 14 | 10 |
| thrombocytopenia | 5 | 5 |
| tremors | 2 | 21 |
| neuropathy | 22 | 15 |
| cramps | 2 | 20 |

The most common grade 3 and 4 adverse events that occurred in 5% or more of the patients included peripheral neuropathy (22%), neutropenia (14%), palmer planter erythrodysthesia (8%), and thrombocytopenia (5%). To improve the grade 3 and 4 adverse events, a dose modification schema to sacrifice vincristine was made. For grade 2 neuropathy, the dose of vincristine was reduced by 50%, to 1 mg. For grade 3 or 4 neuropathy, vincristine was withheld from the treatment regimen, and if the toxicity resolved, therapy was restarted at 50% of the initial drug dose.

In all, 464 treatments of chemotherapy were administered, 225 treatments were given with full dose vincristine, and 242 were given with reduced dose or eliminated vincristine. Twenty two patients developed grade 3/4 neuropathy. Those 22 patients experienced 30 events where dose modification of vincristine and or thalidomide resulted in resolution of the grade 3/4 to grade one or total resolution except in 5 patients where the severity dropped by only one grade. Reducing or eliminating vincristine had a significant positive effect on progression free survival and overall survival on univariate analysis (p<0.0001 and=0.005), respectively.

On multivariate analysis where adjustments were made for age at start of study, platelet count, stage, quality of response (CR or near CR versus SD or PR), and or thalidomide dose, the reduction of the vincristine dose or totally eliminating the vincristine continued to benefit patients, with a continued significant improvement in the progression free survival as compared to the group that received the full vincristine dose. This effect is illustrated in FIG. 4, where the survival probability is shown as a function of progression free survival, in months, for the patients treated with Doxil®, dexamethasone, and thalidomide, in the absence of vincristine or with a reduced dose of vincristine (p=0.0121).

### Example 2

Treatment of Multiple Myeloma Patient with Doxorubicin, dexamethasone, and thalidomide

A male patient presented with fatigue and rib pain. On bone marrow biopsy, there were 84.5% plasma cells, diffuse lytic lesions, and serum creatine was elevated at 1.8 mg/dL (normal 0.8-1.5 mg/dL). Upon diagnosis of multiple myeloma, the subject is treated with four cycles of the following regimen: thalidomide by mouth every night without food on days 1-28, with dosing gradually increasing during cycle 1 as follows: 50 mg on days 1-7, 100 mg on days 8-14, 150 mg on days 15-21, and 200 mg on days 22-28, with 200 mg being given daily thereafter for all subsequent cycles; dexamethasone is given at 40 mg by mouth on days; 1-4, days 9-12 and days 17-20; Doxil® is administered on day 1 via intravenous infusion of 40 mg/m² over 60 minutes. The cycle is repeated every 28 days, for a total of four cycles.

### Example 3

Treatment of Multiple Myeloma Patient with Doxorubicin, Dexamethasone, and Thalidomide and Autologous Stem Cell Transplantation

A female patient presents with fatigue and other symptoms of anemia. Initial bone marrow biopsy demonstrates 50% plasma cells. The subject is treated with the following regimen: thalidomide by mouth every night without food on days 1-28, with dosing gradually increasing to 300 mg daily; dexamethasone is given at 40 mg by mouth on days 1-4, days 9-12 and days 17-20; Doxil® administered on day 1 via intravenous infusion of 40 mg/m² over 60 minutes. The cycle is repeated every 28 days, for a total of six cycles.

Upon completing the six cycles, the patient shows a very good partial response and undergoes a stem cell mobilization and autotransplant. Stem cell mobilization consists of cyclophosphamide (4.5 g/m²) and GM-CSF, for a collection of a minimum of 2×10⁶ CD34+cells for peripherial blood stem cell transplantation. During induction, the patient continues to take thalidomide and melphalan (140-200 mg/m²) is given as conditioning. The patient engrafts within 12-16 days after transplantation.

### Example 4

Treatment of Multiple Myeloma Patient with Doxorubicin, Dexamethasone, and Thalidomide and Autologous Stem Cell Transplantation

A female patient presents with fatigue and other symptoms of anemia. Initial bone marrow biopsy demonstrates 50% plasma cells. The subject is treated with the following regimen: thalidomide by mouth every night without food on days 1-28, with dosing gradually increasing to 300 mg daily; dexamethasone is given at 40 mg by mouth on days 1-4, days 9-12 and days 17-20; daunorubicin entrapped in liposomes having an outer surface coating of poly(ethylene glycol) with anti CD19 antibodies attached to distal ends of the polymer chains, administered on day 1 via intravenous infusion of 40 mg/M² over 60 minutes. The cycle is repeated every,28 days, for a total of three cycles.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method for treating multiple myeloma, comprising:
administering a combination of chemotherapeutic agents consisting essentially of an anthracycline in liposome-entrapped form, dexamethasone, thalidomide, and a dose of vincrstine less than a recommended dose for treatment of multiple myeloma.

2. The method according to claim 1, wherein said administering is prior to autologous stem cell transplant.

3. The method according to claim 1, wherein said administering is prior to or concurrent with an induction therapy regimen to mobilize stem cell production.

4. The method according to claim 1, wherein said liposome-entrapped anthracycline is liposome-entrapped daunorubicin.

5. The method according to claim 1, wherein said liposome-entrapped anthracycline is liposome-entrapped doxorubicin.

6. The method according to claim 5, wherein said liposome-entrapped doxorubicin is comprised of liposomes having an external coating of a hydrophilic polymer.

7. The method according to claim 6, wherein said hydrophilic polymer is poly(ethylene glycol).

8. The method according to claim 6, wherein said liposomes comprise a ligand for targeting the liposomes to a B-cell or a T-cell.

9. The method according to claim 8, wherein said ligand is selected from the group consisting of an anti-CD19 antibody, an anti-CD20 antibody, an anti-CD22 antibody, an anti-CD4 antibody, and an anti-CD8 antibody.

10. The method according to claim 1, wherein said thalidomide is administered at a dose of at least about 50 mg/day.

11. The method according to claim 1, wherein said dexamethasone is administered orally.

12. The method according to claim 1, wherein said dexamethasone is at a dose of at least about 40 mg.

13. The method according to claim 1, wherein said administering further comprises administering said combination once every four weeks for at least about three months.

14. The method according to claim 1, wherein said administering further comprises administering said combination once every four weeks for at least about six months.

15. The method of claim 13, further comprising the step of administering prednisone subsequent to said administering once every four weeks for at least about three months.

16. The method of claim 14, further comprising the step of administering prednisone subsequent to said administering once every four weeks for at least about six months.

17. An improvement in a method of treating multiple myeloma by treatment with liposome-entrapped doxorubicin, vincristine, dexamethasone, and thalidomide, comprising:
administering, in the absence of vincristine, doxorubicin in liposome-entrapped form, dexamethasone, and thalidomide.

18. A method for treating multiple myeloma, comprising:
administering, in the absence of vincristine, doxorubicin in liposome-entrapped form, dexamethasone, and thalidomide.

19. A method for treating multiple myeloma, comprising administering a combination of chemotherapeutic agents consisting essentially of:
(a) doxorubicin entrapped in liposomes, the doxorubicin administered intravenously at a dose of at least about 40 mg/m²;
(b) dexamethasone administered orally at dose of at least about 40 mg,
(c) thalidomide administered orally at a dose of at least about 50 mg.

20. A method for treating multiple myeloma, comprising:
administering over a 28-day treatment cycle a combination of chemotherapeutic agents consisting essentially of: (a) doxorubicin entrapped in liposomes, administered intravenously at a dose of at least about 40 mg/m² on day one of the treatment cycle; (b) dexamethasone, administered orally at dose of at least about 40 mg on days 1-4, 9-12 and 17-20 of the treatment cycle; and (c) thalidomide administered orally at a dose of at least about 50 mg per day; and
repeating said administering between 4-12 times.
